(19) United States
(12) Patent Application Publication
(10) Pub. No.: US 2026/0131083 A1
(43) Pub. Date: May 14, 2026

(54) MANUAL SYRINGE HAVING FLEXIBLE PLUNGER

(71) Applicant: LABORATOIRES VIVACY, Paris (FR)

(72) Inventors: Emma Rochedix, Saint Julien en Genevois (FR); Pierre Suc, Annecy (FR); Safia Mouldi, Saint-Genis-Pouilly (FR)

(21) Appl. No.: 19/119,073

(22) PCT Filed: Sep. 25, 2023
(86) PCT No.: PCT/EP2023/076395

§ 371 Date: Apr. 7, 2025

(30) Foreign Application Priority Data
Oct. 6, 2022 (FR) FR2210240

(51) Int. Cl. A61M 5/315

(52) U.S. Cl. CPC A61M 5/31578; A61M 5/31511; A61M 2005/31518; A61M 2205/0216; A61M 2205/586

(57) ABSTRACT

A manual syringe includes syringe body; gripping wings; a piston rod, mounted so as to be able to slide inside the syringe body; a stopper; and a manual pusher, on to which an injection pressure is manually exerted. The manual pusher includes rigid part and a deformable part. The deformable part is designed to bend toward the piston rod, according to a deflection of between 5% and 25% of the length of the piston rod, when the injection pressure is manually exerted on the manual pusher.

### CROSS-REFERENCE TO RELATED APPLICATIONS

This application is a national phase entry under 35 U.S.C. § 371 of International Patent Application PCT/EP2023/076395, filed Sep. 25, 2023, designating the United States of America and published as International Patent Publication WO 2024/074329 A1 on Apr. 11, 2024, which claims the benefit, under Article 8 of the Patent Cooperation Treaty, of French Patent Application Serial No. FR 2210240, filed Oct. 6, 2022.

### TECHNICAL FIELD

The disclosure relates to the technical field of manual syringes, i.e., syringes having a pusher on which an injection pressure is manually exerted by a health professional.

The disclosure notably finds an application in the injection of viscoelastic gels (e.g., based on hyaluronic acid), for example in the field of anti-aging and medical esthetics.

### BACKGROUND

A manual syringe known from the prior art has:
a syringe body, intended to receive a product to be injected; the syringe body having a first end, intended to be equipped with a needle holder, and an opposite second end;
gripping wings, arranged at the second end of the syringe body;
a piston rod, mounted so as to be able to slide inside the syringe body, the piston rod having opposite first and second ends; the piston rod having a length;
a stopper, arranged at the first end of the piston rod; and
a manual pusher, arranged at the second end of the piston rod, on which an injection pressure suitable for injecting the product is manually exerted.

Such a prior art manual syringe is not entirely satisfactory in the case in which the product to be injected requires considerable pressure to be exerted on the pusher. This is notably the case when the product to be injected is a viscoelastic gel based on hyaluronic acid, in which case, it is sometimes necessary to apply a force greater than 50 N. The health professional (e.g., physician) is therefore liable to feel some discomfort, or even pain, when repeatedly using such a prior art syringe. For the same volume, the manual syringe is generally designed to lengthen and narrow the syringe body and the piston rod in order to reduce the contact surface area with the product to be injected, and thereby reduce the viscosity force. However, lengthening and narrowing the piston rod also lead to discomfort for the health professional. This discomfort in gripping the syringe is particularly evident at the beginning of injection, when the second end of the piston rod is situated at a significant distance from the gripping wings.

In addition, the product can be injected into the wrinkles of a patient, more particularly under the eyes. Such an injection therefore requires excellent stability in order to obtain the desired precision (which can be of the order of ±1 mm) and reach the wrinkles of the patient effectively and safely. As mentioned above, lengthening and narrowing the piston rod lead to discomfort in gripping the syringe, which is liable to result in a lack of stability and a loss of precision, most particularly at the beginning of injection.

### BRIEF SUMMARY

Embodiments of the disclosure aim to remedy all or some of the abovementioned drawbacks. To this end, a subject of the disclosure is a manual syringe, having:
a syringe body, intended to receive a product to be injected; the syringe body having a first end, intended to be equipped with a needle holder, and an opposite second end;
gripping wings, arranged at the second end of the syringe body;
a piston rod, mounted so as to be able to slide inside the syringe body, the piston rod having opposite first and second ends; the piston rod having a length;
a stopper, arranged at the first end of the piston rod; and
a manual pusher, arranged at the second end of the piston rod, on which an injection pressure suitable for injecting the product is manually exerted;
notable in that the manual pusher comprises:
   a rigid part; and
   a deformable part, designed to bend toward the piston rod according to a deflection of between 5% and 20% of the length of the piston rod, when the injection pressure is manually exerted on the manual pusher.

The deformable part may be designed to bend toward the piston rod according to a deflection of between 5% and 25% of the length of the piston rod, when the injection pressure is manually exerted on the manual pusher. The deflection is advantageously between 10% and 25%, more preferentially between 15% and 25%, of the length of the piston rod, when the injection pressure is manually exerted on the manual pusher.

Thus, a manual syringe according to embodiments of the disclosure makes it possible, by virtue of such a deformable part, to improve the comfort of the health professional:
(i) during the injection by conforming to the shape of the palm of the hand of the health professional, and
(ii) most particularly at the beginning of injection by bringing the deformable part of the pusher (and therefore the hand of the health professional) closer to the gripping wings.

This results in better stability of the syringe, allowing great precision during the injection.

The manual syringe according to embodiments of the disclosure may have one or more of the following features.

According to one feature of the disclosure, the deformable part is made of a first material having a first Young's modulus; and the deformable part has weakening zones of the first material, arranged as a function of the first Young's modulus to bend the first material toward the piston rod according to the deflection of between 5% and 20% of the length of the piston rod, when the injection pressure is manually exerted on the manual pusher.

The weakening zones of the first material may be arranged as a function of the first Young's modulus to bend the first material toward the piston rod according to a deflection of between 5% and 25% (preferentially between 10% and 25%, more preferentially between 15% and 25%) of the length of the piston rod, when the injection pressure is manually exerted on the manual pusher.

Thus, an advantage obtained by the weakening zones is that a certain freedom in the choice of the first material is allowed. The first material may be a rigid material or a flexible material. The arrangement of the weakening zones (their number, spatial distribution, volume, etc.) is adjusted as a function of the first Young's modulus in order to bend the first material toward the piston rod according to the desired deflection.

According to one feature of the disclosure, each weakening zone of the first material forms a recess in the material, the recess in the material preferably forming a groove within the first material.

Thus, an advantage obtained is the ease of manufacture of such weakening zones, for example by molding.

According to one feature of the disclosure, the weakening zones of the first material are arranged to form an array of periodic patterns.

Thus, an advantage obtained is that the spatial extent of the weakening zones is optimized in order to obtain the desired deflection.

According to one feature of the disclosure:
the deformable part of the manual pusher is able to move between a rest position and a bent position in which the deformable part is bent toward the piston rod according to the deflection of between 5% and 20% of the length of the piston rod; and
the weakening zones extend along mutually parallel axes when the deformable part is in the rest position.

The deformable part of the manual pusher may be able to move between a rest position and a bent position in which the deformable part is bent toward the piston rod according to a deflection of between 5% and 25% (preferentially between 10% and 25%, more preferentially between 15% and 25%) of the length of the piston rod.

Thus, an advantage obtained is the ease of manufacture of such weakening zones, for example by molding.

According to one feature of the disclosure, the first material is rigid, and the rigid part of the manual pusher is made of the first material.

Thus, an advantage obtained is that the rigid part and the deformable part of the pusher can be made of the same material, and this makes it possible to reduce the operating time for manufacturing the syringe.

According to one feature of the disclosure, the first material is flexible.

Thus, an advantage obtained is that greater freedom is allowed for the arrangement of the weakening zones than when the first material is rigid in order to obtain the desired deflection.

According to one feature of the disclosure, the deformable part of the manual pusher is made of a flexible material having a Young's modulus adapted to bend the flexible material toward the piston rod according to the deflection of between 5% and 20% of the length of the piston rod, when the injection pressure is manually exerted on the manual pusher.

The deformable part of the manual pusher is made of a flexible material having a Young's modulus adapted to bend the flexible material toward the piston rod according to a deflection of between 5% and 25% (preferentially between 10% and 25%, more preferentially between 15% and 25%) of the length of the piston rod, when the injection pressure is manually exerted on the manual pusher.

Thus, an advantage obtained is that the production of weakening zones is dispensed with. In other words, the deformable part of the manual pusher is without weakening zones of the flexible material.

According to one feature of the disclosure:
the piston rod extends along a first longitudinal axis; and
the manual pusher extends along a second longitudinal axis, perpendicular to the first longitudinal axis.

According to one feature of the disclosure:
the deformable part of the manual pusher is able to move between a rest position and a bent position in which the deformable part is bent toward the piston rod according to the deflection of between 5% and 20% of the length of the piston rod; and
the weakening zones of the first material each extend along an axis perpendicular to the first and second longitudinal axes when the deformable part is in the rest position.

The deformable part of the manual pusher may be able to move between a rest position and a bent position in which the deformable part is bent toward the piston rod according to a deflection of between 5% and 25% (preferentially between 10% and 25%, more preferentially between 15% and 25%) of the length of the piston rod.

Thus, an advantage obtained is that the spatial extent of the weakening zones is optimized in order to obtain the desired deflection.

According to one feature of the disclosure:
the deformable part of the manual pusher is able to move between a rest position and a bent position in which the deformable part is bent toward the piston rod according to the deflection of between 5% and 20% of the length of the piston rod; and
the weakening zones of the first material have a longitudinal section, along the first longitudinal axis, defining an inverted U-shape or an inverted V-shape when the deformable part is in the rest position and when the first longitudinal axis is a vertical axis, the second end of the piston rod being situated above the first end of the piston rod with respect to the vertical axis.

The deformable part of the manual pusher may be able to move between a rest position and a bent position in which the deformable part is bent toward the piston rod according to a deflection of between 5% and 25% (preferentially between 10% and 25%, more preferentially between 15% and 25%) of the length of the piston rod.

Thus, an advantage obtained by such shapes is that the spatial extent of the weakening zones is optimized in order to obtain the desired deflection.

According to one feature of the disclosure:
the manual pusher has a length along the second longitudinal axis; and
the weakening zones of the first material have a total length, along the second longitudinal axis, of between 30% and 50% of the length of the manual pusher, when the deformable part is in the rest position.

Thus, an advantage obtained is that a good compromise is obtained in terms of spatial distribution between the rigid part of the manual pusher (necessary for an effective thrust for the injection) and the deformable part of the manual pusher (necessary for the comfort and precision of the injection).

According to one feature of the disclosure:
the manual pusher has an upper part on which an injection pressure suitable for injecting the product is manually exerted, and a lower part opposite the upper part; and
the lower part of the manual pusher has two stop members arranged on either side of the first longitudinal axis so as to block sliding of fingers along the second longitudinal axis.

Thus, an advantage obtained is that better stability of the fingers positioned beneath the manual pusher is obtained, and this is particularly useful during an aspiration test that can be performed by a health professional. An aspiration test consists in verifying that the needle is not in a vein of a patient, in the context of an application of intradermal injection type. To this end, the health professional carries out aspiration by pulling on the pusher, in the direction opposite to the direction of injection of the product, in order to verify the absence of blood reflux. This aspiration test leads to the exertion of a significant force on the lower part of the manual pusher, such that the fingers of the health professional are liable to slide along the second longitudinal axis. Such stop members create a physical obstruction to the fingers positioned on the lower part of the manual pusher, and thus make it possible to make the aspiration test safer by opposing sliding of the fingers along the second longitudinal axis.

According to one feature of the disclosure:
the manual pusher successively has a first lateral zone, a central zone and a second lateral zone along the second longitudinal axis;
the central zone forms the rigid part; and
the first lateral zone and/or the second lateral zone form the deformable part.

Thus, an advantage obtained is that it is possible to conform to the shape of the palm of the hand of the health professional during the injection.

According to one feature of the disclosure:
the manual pusher has a central zone and a peripheral zone extending around the central zone;
the central zone forms the rigid part; and
the peripheral zone forms the deformable part.

Thus, an advantage obtained is that it is possible to conform to the shape of the palm of the hand of the health professional during the injection.

According to one feature of the disclosure, the manual pusher is constituted of a piston head formed at the second end of the piston rod, the piston head comprising the rigid part and the deformable part.

Thus, an advantage obtained is that the operating time for manufacturing the syringe can be reduced.

According to one feature of the disclosure, the manual pusher has:
a rigid piston head formed at the second end of the piston rod; and
a gripping member, arranged to cover the piston head.

Thus, an advantage obtained by the gripping member is that the comfort of the health professional during the injection can be improved by avoiding direct contact with the rigid piston head.

According to one feature of the disclosure:
the gripping member has a rigid zone and a deformable zone;
the rigid part of the manual pusher is formed by the piston head and the rigid zone of the gripping member; and
the deformable part of the manual pusher is formed by the deformable zone of the gripping member.

According to one feature of the disclosure:
the gripping member has a deformable zone;
the rigid part of the manual pusher is formed by the piston head; and
the deformable part of the manual pusher is formed by the deformable zone of the gripping member.

According to one feature of the disclosure, the length of the piston rod is between mm and 120 mm.

### DEFINITIONS

“Rigid part” is understood to mean a part of the pusher that is rigid (i.e., non-deformable) under normal conditions of use, for example at ambient temperature (between 20° C. and 30° C.) and under atmospheric pressure.
“Deformable part” is understood to mean a part of the pusher that is designed to deform under normal conditions of use, for example at ambient temperature (between 20° C. and 30° C.) and under atmospheric pressure.
“Bend” is understood to mean the capacity to deform via a deformation of bending type, i.e., the deformation obtained by the action of a load resulting in a curvature. Deformation of bending type can be elastic, i.e., the deformable part can return to its original shape after having been deformed (reversible deformation). Deformation of bending type can be plastic (irreversible), in which case the manual syringe is a single-use syringe.
“Deflection” is understood to mean the maximum value of the displacement of the deformable part along an axis parallel to the longitudinal axis of the piston rod.
The values X and Y expressed using the expressions “between X and Y” or “of between X and Y” are included in the defined range of values.
“Young's modulus” is understood to mean the Young's modulus stricto sensu or the modulus at 100% when the first material is an elastomer.
“Array of periodic patterns” is understood to mean patterns spaced apart according to a regular distance interval (spatial period) when the deformable part is in the rest position.
“Flexible” is understood to mean that the first material is flexible under normal conditions of use, for example at ambient temperature (between 20° C. and 30° C.) and under atmospheric pressure.
“Inverted U-shape” is understood to mean the symmetrical image of a “U” shape with respect to a horizontal axis.
“Inverted V-shape” is understood to mean the symmetrical image of a “V” shape with respect to a horizontal axis.
“Constituted” is understood to mean that the piston head is the one and only element making up the manual pusher.

### BRIEF DESCRIPTION OF THE DRAWINGS

Other features and advantages will become apparent from the detailed description of various embodiments of the disclosure, the description containing examples and references to the appended drawings.

FIG. 1 is a schematic exploded perspective view of a manual syringe according to embodiments of the disclosure.

FIG. 1bis a schematic view similar to FIG. 1, in which the rigid part of the manual pusher is formed by the piston head.

FIG. 2 is a schematic perspective view of a manual syringe according to embodiments of the disclosure, illustrating a deformable part of the manual pusher in the rest position.

FIG. 3 is a schematic view similar to FIG. 2, illustrating the deformable part of the manual pusher in the bent position.

FIG. 4 is a schematic view similar to FIG. 3, the deformable part being viewed from another angle.

FIG. 5 is a partial schematic perspective view of a manual syringe according to embodiments of the disclosure, illustrating a piston rod and a manual pusher.

FIG. 6 is a schematic view on an enlarged scale of the piston rod and manual pusher illustrated in FIG. 5.

FIG. 7 is a partial schematic perspective view of a manual syringe according to embodiments of the disclosure, illustrating a piston rod and a manual pusher made of different materials, the deformable part of the manual pusher being in the rest position.

FIG. 8 is a schematic view similar to FIG. 7, the deformable part of the manual pusher being in the bent position.

FIG. 9 is a partial schematic exploded perspective view of a manual syringe according to embodiments of the disclosure, illustrating a piston head and a gripping member arranged to cover the piston head.

FIG. 10 is a schematic view in cross section illustrating the deformable part of the manual pusher in the rest position and in the bent position.

FIG. 11 is a schematic perspective view on an enlarged scale of a manual pusher having a deformable part in the rest position.

FIG. 12 is a schematic perspective view of a gripping member intended to cover a piston head, the gripping member having a deformable zone in the rest position.

FIG. 13 is a schematic perspective view of gripping wings.

FIG. 14 is a schematic perspective view illustrating the fastening of the gripping wings to the second end of the syringe body.

FIG. 15 is a schematic perspective view illustrating the insertion of the piston rod through the gripping wings.

FIG. 16 is a schematic top view of a manual pusher, illustrating a rigid central zone and two deformable lateral zones.

FIG. 17 is a schematic top view of a manual pusher, illustrating a rigid central zone and a deformable peripheral zone.

FIG. 18 is a partial schematic perspective view of a manual syringe according to embodiments of the disclosure, illustrating a lower part of a manual pusher provided with stop members.

It should be noted that, for the sake of legibility and ease of understanding, the drawings described above are schematic and are not necessarily to scale.

### DETAILED DESCRIPTION

For the sake of simplicity, elements that are identical or that perform the same function in the various embodiments will bear the same references.

A subject of the disclosure is a manual syringe, having:
a syringe body 1, intended to receive a product to be injected; the syringe body 1 having a first end 10, intended to be equipped with a needle holder, and an opposite second end 11;
gripping wings 2, arranged at the second end 11 of the syringe body 1;
a piston rod 3, mounted so as to be able to slide inside the syringe body 1, the piston rod 3 having opposite first and second ends 30, 31; the piston rod 3 having a length L₁;
a stopper 4, arranged at the first end 30 of the piston rod 3; and
a manual pusher 5, arranged at the second end 31 of the piston rod 3, on which an injection pressure suitable for injecting the product is manually exerted;
notable in that the manual pusher 5 comprises:
   a rigid part 50; and
   a deformable part 51, designed to bend toward the piston rod 3 according to a deflection δ of between 5% and 20% of the length L₁ of the piston rod 3, when the injection pressure is manually exerted on the manual pusher 5.

The deformable part 51 may be designed to bend toward the piston rod 3 according to a deflection δ of between 5% and 25% of the length L₁ of the piston rod 3, when the injection pressure is manually exerted on the manual pusher 5. The deflection δ is advantageously between 10% and 25%, more preferentially between 15% and 25%, of the length L₁ of the piston rod 3, when the injection pressure is manually exerted on the manual pusher 5.

### Syringe Body

The syringe body 1 is intended to receive a product to be injected. The product to be injected is preferably a viscous product, more preferentially a viscoelastic gel, even more preferentially a viscoelastic gel based on hyaluronic acid. The syringe body 1 is preferably cylindrical.

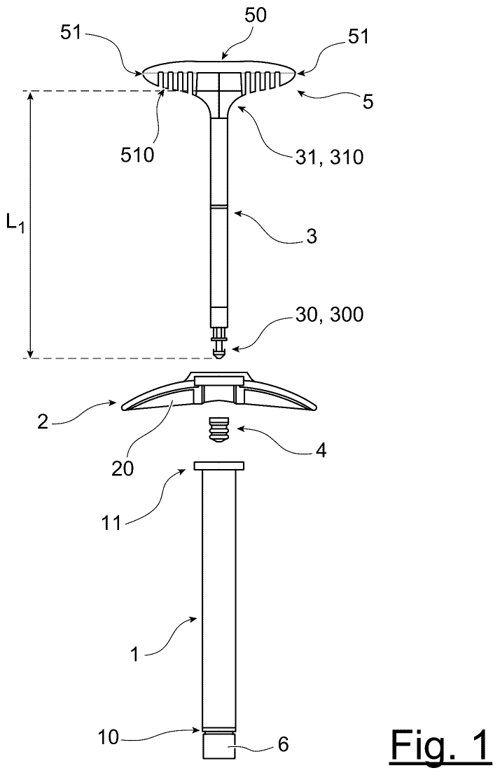

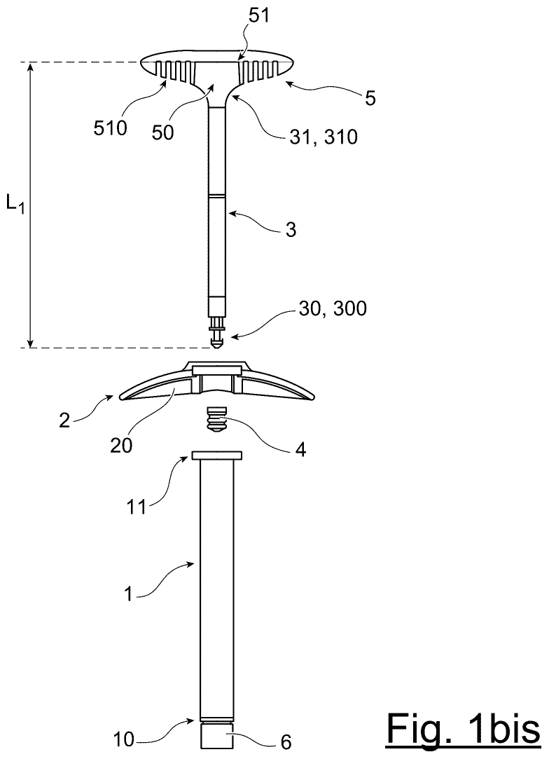

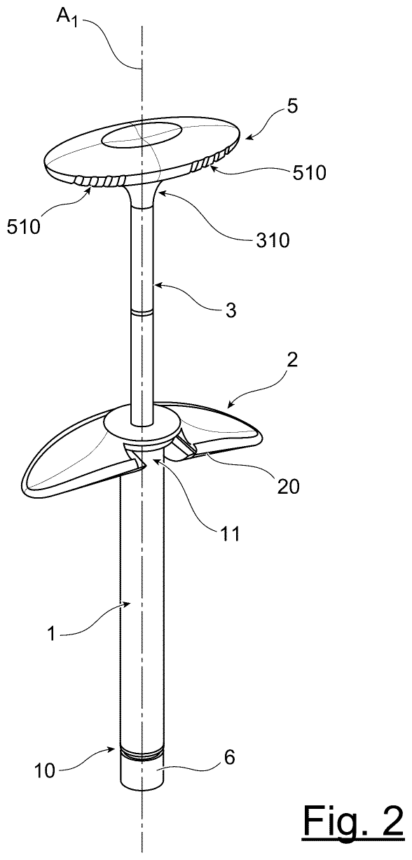

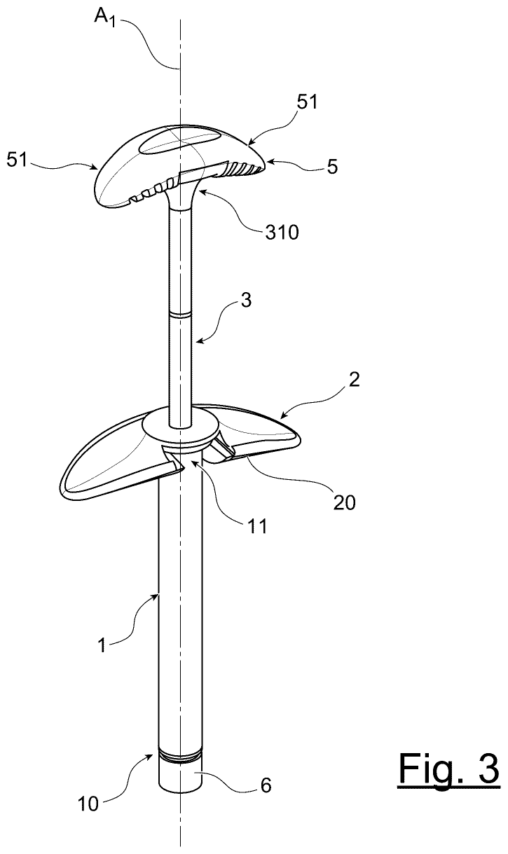

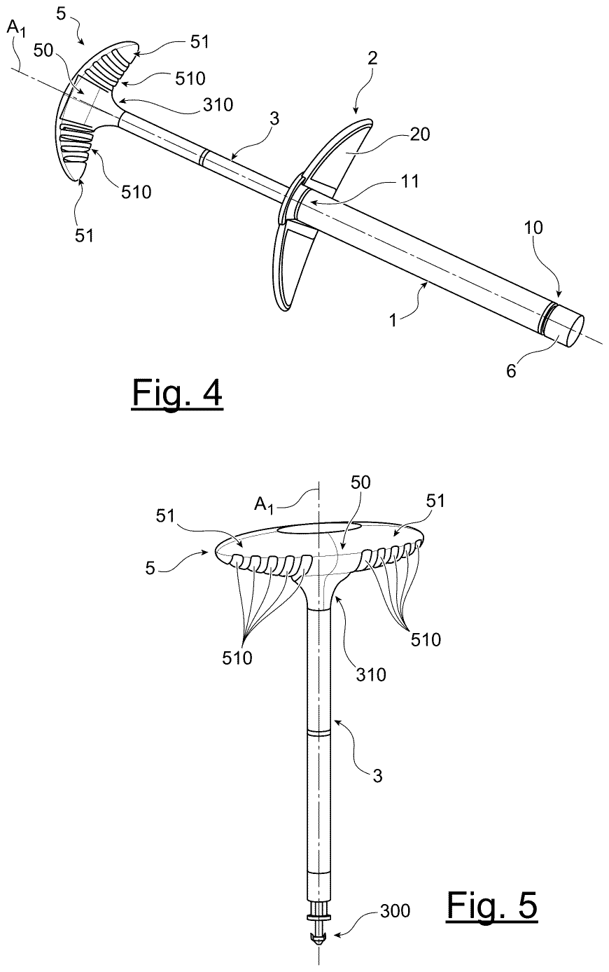

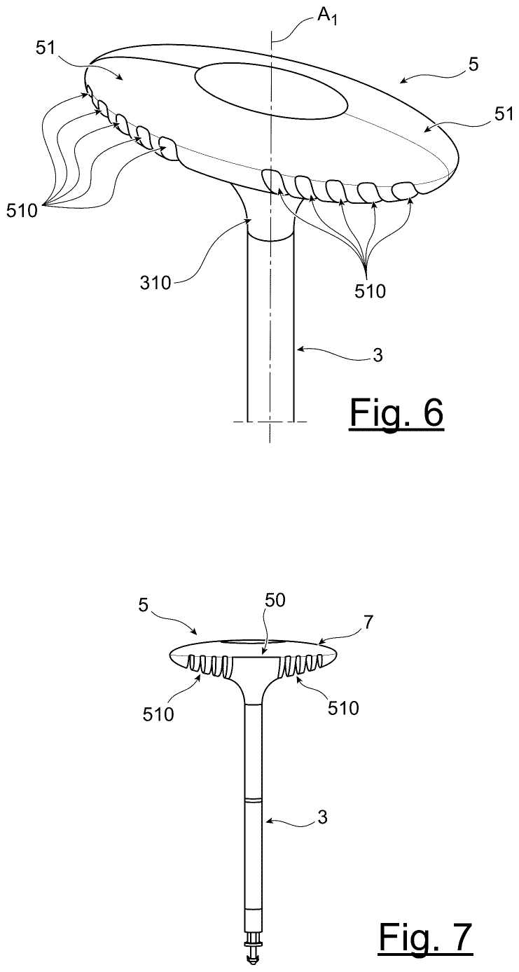

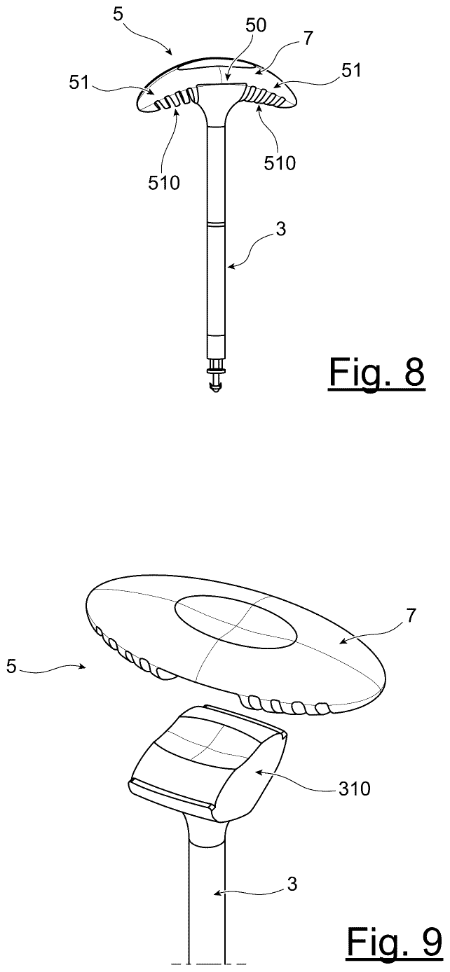

The syringe body 1 has a first end 10 intended to be equipped with a needle holder. The syringe body 1 has a second end 11, opposite the first end 10. The first and second ends 10, 11 of the syringe body 1 are open. The first end 10 of the syringe body 1 is open so as to receive the needle holder. As illustrated in FIGS. 1 to 4, in the absence of a needle holder, the first end 10 of the syringe body 1 may be obstructed by a cap 6. The second end 11 of the syringe body 1 is open so as to receive the piston rod 3.

The syringe body 1 is advantageously made of a first plastics material, the first plastics material preferably being polycarbonate.

### Gripping Wings

The gripping wings 2 are arranged at the second end 11 of the syringe body 1. As illustrated in FIG. 14, the gripping wings 2 can be mounted at the second end 11 of the syringe body 1 via an embedded connection.

The gripping wings 2 have a gripping surface 20 advantageously having an area of between 400 mm² and 900 mm² , preferably of between 700 mm² and 800 mm² . The gripping surface 20 is advantageously concave, oriented toward the syringe body 1.

The gripping wings 2 form lateral parts of the syringe projecting from the syringe body 1. The gripping wings 2 are advantageously in one piece. The gripping wings 2 extend along a longitudinal axis, and advantageously have a transverse profile (with respect to the longitudinal axis) forming a convex surface.

The gripping wings 2 are advantageously made at least partially of the first plastics material. The gripping surface 20 of the gripping wings 2 is advantageously made at least partially of a second plastics material, different from the first plastics material. The second plastics material advantageously has a hardness of between 30 Shore A and 70 Shore A, preferably between 50 Shore A and 70 Shore A. By way of non-limiting examples, the second plastics material may be a thermoplastic of SEBS type, i.e., polystyrene-b-poly(ethylene-butylene)-b-polystyrene, or silicone.

### Piston Rod

The piston rod 3 is mounted so as to be able to slide inside the syringe body 1. The piston rod 3 has opposite first and second ends 30, 31. The piston rod 3 advantageously has a snap-fastening head 300 arranged at the first end 30 of the piston rod 3. The piston rod 3 advantageously has a piston head 310 arranged at the second end 31 of the piston rod 3. The piston head 310 and the piston rod 3 are advantageously in one piece. The piston rod 3 is preferentially made of a plastics material, more preferentially a thermoplastic material. By way of non-limiting example, the piston rod 3 may be made of polycarbonate.

The piston rod 3 extends along a first longitudinal axis A₁. The length L₁ of the piston rod 3 (along the first longitudinal axis A1) is preferentially between 40 mm and 120 mm. As illustrated in FIG. 1, the length L₁ of the piston rod 3 is defined between the end of the piston head 310 and the end of the snap-fastening head 300. The ratio between the width of the piston rod 3 and the length L₁ of the piston rod 3 may be between 2% and 30%, preferentially between 3% and 20%, more preferentially between 4% and 10%, even more preferentially between 5.5% and 8.5%.

### Stopper

The stopper 4 (illustrated in FIG. 1) is arranged at the first end 30 of the piston rod 3. The stopper 4 may have an elastically deformable internal wall delimiting a cavity. “Internal wall” is understood to mean the internal surface of the stopper 4 delimiting a cavity, the stopper 4 being hollow. “Elastically deformable” is understood to mean that the internal wall of the stopper 4 can deform in a reversible manner under the action of mechanical stresses (lower than the elastic limit), i.e., the internal wall of the stopper 4 can return to its initial shape when the mechanical stresses cease. The snap-fastening head 300 is advantageously snap-fastened inside the cavity of the stopper 4. “Snap-fastened” is understood to mean an elastic engagement between the snap-fastening head 300 and the internal wall of the stopper 4, inside the cavity of the stopper 4. To this end, the snap-fastening head 300 may have:
a flat bearing surface, coming to bear against the internal wall of the stopper 4 when the piston rod 3 slides in the direction opposite to the direction of injection of the product; and
at least one projecting element, projecting from the flat bearing surface, arranged to penetrate into the internal wall of the stopper 4 when the piston rod 3 slides in the direction opposite to the direction of injection of the product.

The stopper 4 is preferentially made of an elastomer material, preferably a chlorinated or brominated butyl rubber.

### Manual Pusher

The manual pusher 5 is arranged at the second end 31 of the piston rod 3. An injection pressure is manually exerted on the manual pusher 5 by the health professional, the injection pressure being suitable for injecting the product.

The manual pusher 5 comprises:
a rigid part 50; and
a deformable part 51, designed to bend toward the piston rod 3 according to a deflection δ (illustrated in FIG. 10) of between 5% and 20% of the length L₁ of the piston rod 3, when the injection pressure is manually exerted on the manual pusher 5.

The deformable part 51 may be designed to bend toward the piston rod 3 according to a deflection δ of between 5% and 25% of the length L₁ of the piston rod 3, when the injection pressure is manually exerted on the manual pusher 5. The deflection δ is advantageously between 10% and 25%, more preferentially between 15% and 25%, of the length L₁ of the piston rod 3, when the injection pressure is manually exerted on the manual pusher 5.

The deflection δ is the maximum value of the displacement of the deformable part 51 along an axis parallel to the first longitudinal axis A₁ of the piston rod 3.

The manual pusher 5 preferentially extends along a second longitudinal axis A₂, perpendicular to the first longitudinal axis A₁. The manual pusher 5 has a length L₂ along the second longitudinal axis A₂.

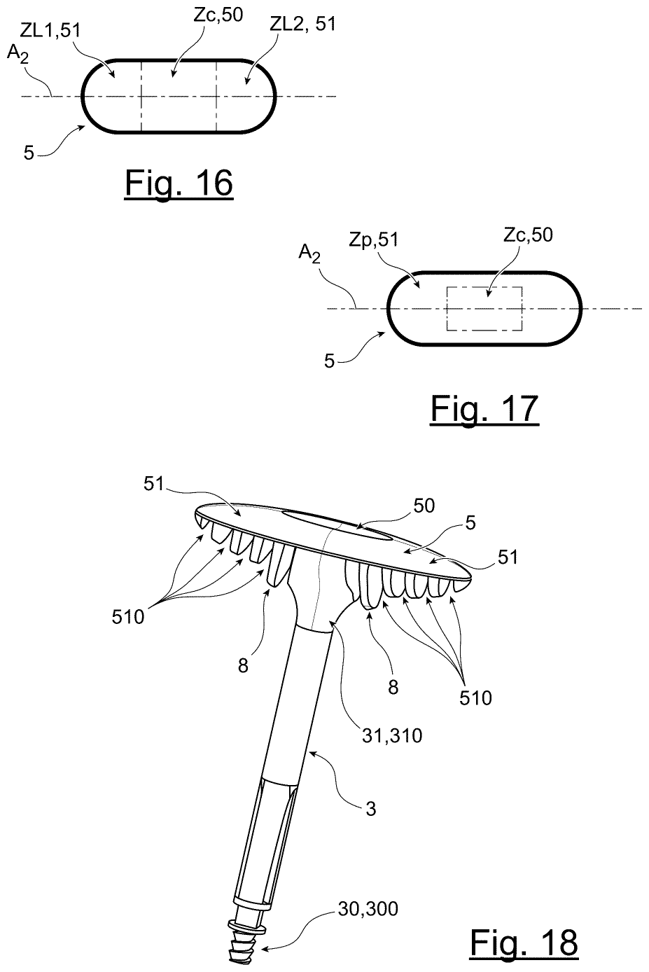

According to a first possibility, illustrated in FIG. 16:
the manual pusher 5 successively has a first lateral zone ZL1, a central zone Zc and a second lateral zone ZL2 along the second longitudinal axis A₂;
the central zone Zc forms the rigid part 50; and
the first lateral zone ZL1 and/or the second lateral zone ZL2 form the deformable part 51.

According to a second possibility, illustrated in FIG. 17:
the manual pusher 5 has a central zone Zc and a peripheral zone Zp extending around the central zone Zc;
the central zone Zc forms the rigid part 50; and
the peripheral zone Zp forms the deformable part 51.

The manual pusher 5 may have:
an upper part on which an injection pressure suitable for injecting the product is manually exerted; and
a lower part, opposite the upper part.

The term “upper” is chosen by convention to denote the part on which an injection pressure is manually exerted to inject the product, regardless of the gripping position. Specifically, in the majority of the gripping positions, the product is injected downward. It should be noted that in certain gripping positions the product can be injected upward or else laterally.

The upper part of the manual pusher 5 advantageously has a curved surface designed to conform to a palmar shape of a hand. The curved surface, designed to conform to a palmar shape of a hand, advantageously has:
a rigid central part; and
at least one deformable lateral part or one deformable peripheral part.

The deformable part 51 of the manual pusher 5 is able to move between a rest position and a bent position in which the deformable part 51 is bent toward the piston rod 3 according to the deflection δ of between 5% and 20% of the length L₁ of the piston rod 3. The deformable part 51 of the manual pusher 5 may be able to move between a rest position and a bent position in which the deformable part 51 is bent toward the piston rod 3 according to a deflection δ of between 5% and 25% of the length L₁ of the piston rod 3. The deflection δ is advantageously between 10% and 25%, more preferentially between 15% and 25%, of the length L₁ of the piston rod 3, when the injection pressure is manually exerted on the manual pusher 5. The deformable part 51 of the manual pusher 5 and the rigid part 50 of the manual pusher 5 remain in contact when the deformable part 51 of the manual pusher 5 is moved from the rest position to the bent position, and from the bent position to the rest position. In particular, the deformable part 51 of the manual pusher 5 and the rigid part 50 of the manual pusher 5 are in contact when the deformable part 51 of the manual pusher 5 is in the rest position. The deformable part 51 of the manual pusher 5 and the rigid part 50 of the manual pusher 5 can be contiguous in a junction plane when the deformable part 51 is in the rest position, the junction plane being perpendicular to the first longitudinal axis A₁. In other words, the deformable part 51 of the manual pusher 5 and the rigid part 50 of the manual pusher 5 are without a spacing distance extending along the first longitudinal axis A₁. More specifically, the deformable part 51 of the manual pusher 5 and the rigid part 50 of the manual pusher 5 are without a spacing distance extending along the first longitudinal axis A₁ whatever the position of the deformable part 51 of the manual pusher 5.

### Embodiment No. 1

### Presence of Weakening Zones

The deformable part 51 is made of a first material having a first Young's modulus. The deformable part 51 has weakening zones 510 of the first material, arranged as a function of the first Young's modulus to bend the first material toward the piston rod 3 according to the deflection δ of between 5% and 20% of the length L₁ of the piston rod 3, when the injection pressure is manually exerted on the manual pusher 5. The weakening zones 510 of the first material may be arranged as a function of the first Young's modulus to bend the first material toward the piston rod 3 according to a deflection δ of between 5% and 25% (preferentially between 10% and 25%, more preferentially between 15% and 25%) of the length L₁ of the piston rod 3, when the injection pressure is manually exerted on the manual pusher 5.

The deformable part 51 of the manual pusher 5 is able to move between a rest position and a bent position in which the deformable part 51 is bent toward the piston rod 3 according to the deflection δ of between 5% and 20% of the length L₁ of the piston rod 3. The deformable part 51 of the manual pusher 5 may be able to move between a rest position and a bent position in which the deformable part 51 is bent toward the piston rod 3 according to a deflection δ of between 5% and 25% of the length L₁ of the piston rod 3. The deflection δ is advantageously between 10% and 25%, more preferentially between 15% and 25%, of the length L₁ of the piston rod 3, when the injection pressure is manually exerted on the manual pusher 5.

Each weakening zone 510 of the first material advantageously forms a recess in the material, the recess in the material preferably forming a groove within the first material.

The weakening zones 510 of the first material are advantageously arranged to form an array of periodic patterns. More specifically, the weakening zones 510 of the first material are advantageously arranged to form an array of periodic patterns when the deformable part 51 of the manual pusher 5 is in the rest position. The number of periodic patterns, the spatial period of the periodic patterns and the spatial distribution of the periodic patterns are adjusted as a function of the first Young's modulus in order to obtain the deflection δ toward the piston rod 3, the deflection δ being between 5% and 20% of the length L₁ of the piston rod 3, when the injection pressure is manually exerted on the manual pusher 5. The number of periodic patterns, the spatial period of the periodic patterns and the spatial distribution of the periodic patterns are adjusted as a function of the first Young's modulus in order to obtain the deflection δ toward the piston rod 3, the deflection δ being able to be between 5% and 25% of the length L₁ of the piston rod 3, when the injection pressure is manually exerted on the manual pusher 5. The deflection δ is advantageously between 10% and 25%, more preferentially between 15% and 25%, of the length L₁ of the piston rod 3, when the injection pressure is manually exerted on the manual pusher 5.

As illustrated in FIG. 11, the weakening zones 510 advantageously extend along mutually parallel axes A when the deformable part 51 is in the rest position.

The weakening zones 510 of the first material each advantageously extend along an axis A perpendicular to the first and second longitudinal axes A₁, A₂ when the deformable part 51 is in the rest position.

The weakening zones 510 of the first material have a longitudinal section, along the first longitudinal axis A₁, advantageously defining an inverted U-shape or an inverted V-shape when the deformable part 51 is in the rest position and when the first longitudinal axis A₁ is a vertical axis, the second end 31 of the piston rod 3 being situated above the first end 30 of the piston rod 3 with respect to the vertical axis.

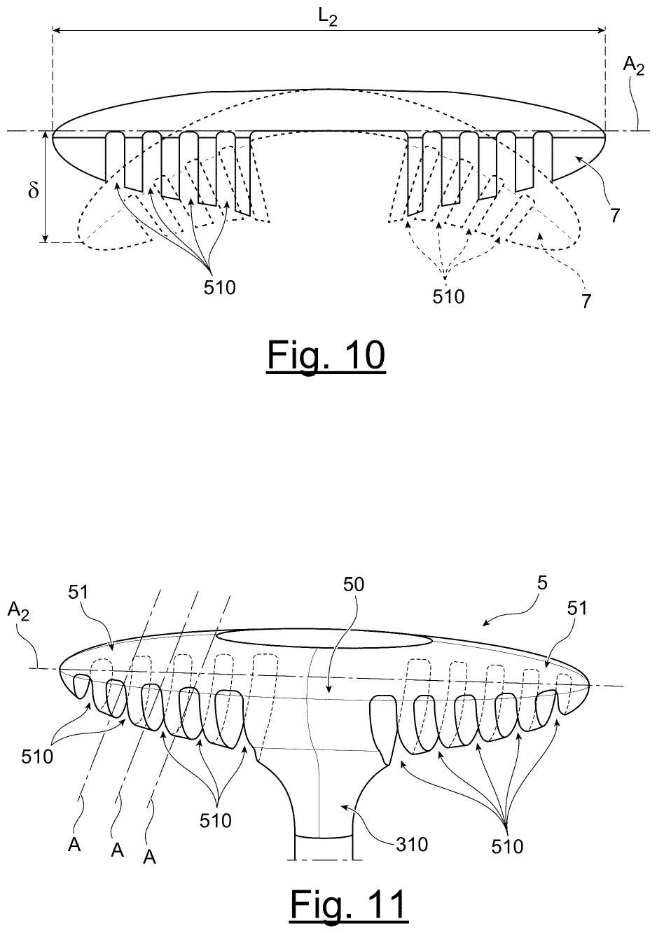

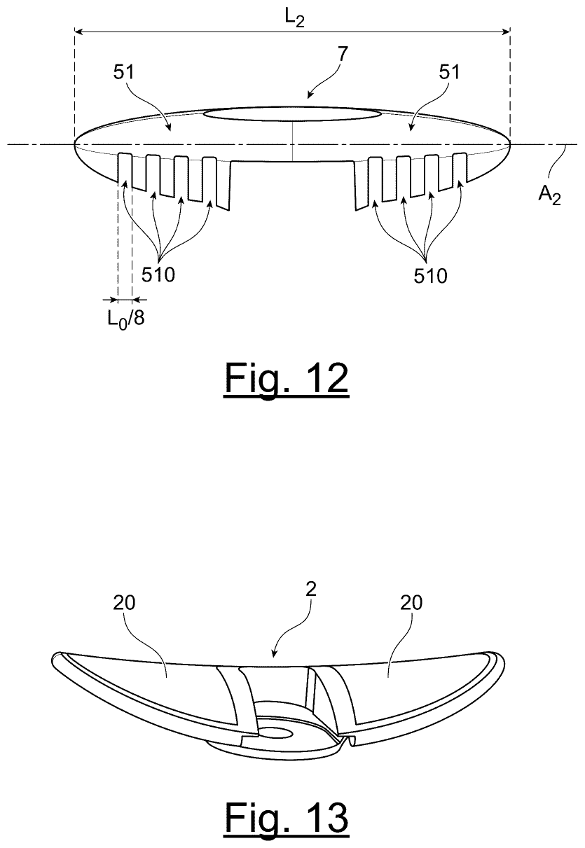

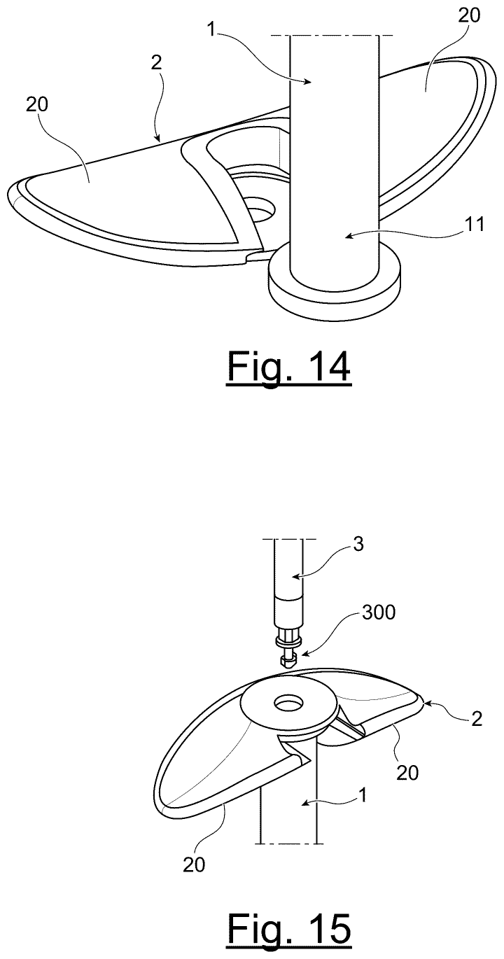

As illustrated in FIG. 12, the weakening zones 510 of the first material have a total length L₀, along the second longitudinal axis A₂, advantageously of between 30% and 50% of the length L₂ of the manual pusher 5, when the deformable part 51 is in the rest position. The total length L₀ of the weakening zones 510, along the second longitudinal axis A₂, corresponds to the cumulative length of the weakening zones 510 (e.g., recesses in the material, hollow periodic patterns). By way of non-limiting example, eight weakening zones 510, of identical length along the second longitudinal axis A₂, are shown in FIG. 12, each weakening zone having a length L₀/8 along the second longitudinal axis A₂. By way of non-limiting example, each weakening zone 510 may have a length along the second longitudinal axis A₂ of between 1 mm and 2 mm.

The first material may be rigid, and the rigid part of the manual pusher 5 is made of the first material. The first rigid material is preferably a polymer, more preferentially a polymer chosen from polypropylene, acrylonitrile butadiene styrene, a polyamide and polyethylene. The first rigid material preferentially has a Young's modulus of between 900 MPa and 1600 MPa.

According to a variant embodiment, the first material is flexible. The first flexible material is preferably an elastomer, more preferentially a thermoplastic elastomer. The first flexible material is, for example, chosen from a rubber, polychloroprene (Neoprene™), silicone and nylon. The first flexible material preferentially has a modulus at 100% of between 1 MPa and 2 MPa.

### Embodiment No. 2

### Absence of Weakening Zones

The deformable part 51 of the manual pusher 5 is made of a flexible material having a Young's modulus adapted to bend the flexible material toward the piston rod 3 according to the deflection δ of between 5% and 20% of the length L₁ of the piston rod 3, when the injection pressure is manually exerted on the manual pusher 5. The deformable part 51 of the manual pusher 5 may be made of a flexible material having a Young's modulus adapted to bend the flexible material toward the piston rod 3 according to a deflection δ of between 5% and 25% (preferentially between 10% and 25%, more preferentially between 15% and 25%) of the length L₁ of the piston rod 3, when the injection pressure is manually exerted on the manual pusher 5. The deformable part 51 of the manual pusher 5 is then without weakening zones 510 of the flexible material. A person skilled in the art can adjust the length of the deformable part 51 of the manual pusher 5 along the second longitudinal axis A₂ as a function of the Young's modulus of the flexible material in order to obtain the deflection δ toward the piston rod 3, the deflection δ being between 5% and 20% of the length L₁ of the piston rod 3, when the injection pressure is manually exerted on the manual pusher 5. A person skilled in the art can adjust the length of the deformable part 51 of the manual pusher 5 along the second longitudinal axis A₂ as a function of the Young's modulus of the flexible material in order to obtain the deflection δ toward the piston rod 3, the deflection δ being between 5% and 25% (preferentially between 10% and 25%, more preferentially between 15% and 25%) of the length L₁ of the piston rod 3, when the injection pressure is manually exerted on the manual pusher 5.

By way of non-limiting examples, the flexible material may be chosen from a polyurethane, expanded polystyrene and cork.

### Embodiment No. 3

### Pusher Constituted of the Piston Head

The manual pusher 5 may be constituted of a piston head 310 formed at the second end 31 of the piston rod 3. The piston head 310 then comprises the rigid part 50 and the deformable part 51 of the manual pusher 5. The manual syringe is then without a gripping member 7 arranged to cover the piston head 310. The piston head 310, constituting the manual pusher 5, ensures the gripping of the syringe with the gripping wings 2.

### Embodiment No. 4

### Pusher Having the Piston Head and a Gripping Member

The manual pusher 5 may have:
a rigid piston head 310 formed at the second end 31 of the piston rod 3; and
a gripping member 7, arranged to cover the piston head 310.

The gripping member 7 and the rigid piston head 310 may be in one piece. According to an alternative, the gripping member 7 can be mounted on the piston head 310 via an embedded connection or an elastic engagement.

According to a first possibility:
the gripping member 7 has a rigid zone and a deformable zone;
the rigid part 50 of the manual pusher 5 is formed by the piston head 310 and the rigid zone of the gripping member 7; and
the deformable part 51 of the manual pusher 5 is formed by the deformable zone of the gripping member 7.

The rigid zone of the gripping member 7 and the rigid piston head 310 may be made of identical or different materials. When the rigid zone of the gripping member 7 and the piston head 310 are made of identical materials, and when the gripping member 7 and the piston head 310 are in one piece, the gripping member 7 and the piston head 310 may be obtained via two-shot (two-material) injection molding, the first material being the material of the piston head 310 and the rigid zone of the gripping member 7, the second material being the material of the deformable zone of the gripping member 7. When the gripping member 7 and the piston head 310 are not in one piece, the gripping member 7 may be obtained via two-shot (two-material) injection molding, the first material being the material of the rigid zone of the gripping member 7, the second material being the material of the deformable zone of the gripping member 7.

According to a second possibility:
the gripping member 7 has a deformable zone;
the rigid part 50 of the manual pusher 5 is formed by the piston head 310; and
the deformable part 51 of the manual pusher 5 is formed by the deformable zone of the gripping member 7.

When the gripping member 7 and the piston head 310 are in one piece, the gripping member 7 and the piston head 310 may be obtained via two-shot (two-material) injection molding, the first material being the material of the piston head 310, the second material being the material of the gripping member 7.

### Stop Members

The lower part of the manual pusher 5 advantageously has two stop members 8 arranged on either side of the first longitudinal axis Ai so as to block sliding of fingers along the second longitudinal axis A₂. The stop members 8 advantageously have a height (dimension along the first longitudinal axis A₁) of between 8 mm and 12 mm. The stop members 8 are advantageously spaced apart from the piston rod 3 by a distance (dimension along the second longitudinal axis A₂) of between 1.5 mm and 2 mm when the deformable part 51 of the manual pusher 5 is in the rest position.

As illustrated in FIG. 18, when the weakening zones 510 form recesses in the material delimiting an alternation of hollow and solid patterns, the stop members 8 advantageously form solid patterns projecting from the adjacent solid patterns so as to block sliding of fingers along the second longitudinal axis A₂. More specifically, the stop members 8 project from the adjacent solid patterns along the first longitudinal axis A₁. The solid patterns advantageously have a height (dimension along the first longitudinal axis A₁) decreasing from the center of the manual pusher 5 toward an edge of the manual pusher 5 (along the second longitudinal axis A₂).

The invention is not limited to the disclosed embodiments. A person skilled in the art will be able to consider the technically workable combinations thereof, and to substitute equivalents therefor.

1. A manual syringe, comprising:
a syringe body, intended to receive a product to be injected; the syringe body having a first end, intended to be equipped with a needle holder, and a second end opposite the first end;
gripping wings, arranged at the second end of the syringe body;
a piston rod, mounted so as to be able to slide inside the syringe body, the piston rod having a first end opposite a second end; the piston rod having a length;
a stopper, arranged at the first end of the piston rod; and
a manual pusher, arranged at the second end of the piston rod, on which manual pusher an injection pressure suitable for injecting the product is manually exertable;
wherein the manual pusher comprises:
a rigid part; and
a deformable part, designed to bend toward the piston rod according to a deflection of between 5% and 25% of the length of the piston rod, when the injection pressure is manually exerted on the manual pusher.

2. The manual syringe of claim 1, wherein the deformable part is made of a first material having a first Young's modulus; and the deformable part comprises weakening zones of the first material, arranged as a function of the first Young's modulus to bend the first material toward the piston rod according to the deflection of between 5% and 25% of the length of the piston rod, when the injection pressure is manually exerted on the manual pusher.

3. The manual syringe of claim 2, wherein each of the weakening zones of the first material forms a recess in the first material, the recess in the first material preferably forming a groove within the first material.

4. The manual syringe of claim 2, wherein the weakening zones of the first material are arranged to form an array of periodic patterns.

5. The manual syringe of claim 2, wherein:
the deformable part of the manual pusher is able to move between a rest position and a bent position in which the deformable part is bent toward the piston rod according to the deflection of between 5% and 25% of the length of the piston rod; and
the weakening zones extend along mutually parallel axes when the deformable part is in the rest position.

6. The manual syringe of claim 2, wherein the first material is rigid, and the rigid part of the manual pusher is made of the first material.

7. The manual syringe of claim 2, wherein the first material is flexible.

8. The manual syringe of claim 1, wherein the deformable part of the manual pusher is made of a flexible material having a Young's modulus adapted to bend the flexible material toward the piston rod according to the deflection of between 5% and 25% of the length of the piston rod when the injection pressure is manually exerted on the manual pusher.

9. The manual syringe of claim 1, wherein:
the piston rod extends along a first longitudinal axis; and
the manual pusher extends along a second longitudinal axis perpendicular to the first longitudinal axis

10. The manual syringe of claim 9, wherein:
the deformable part is made of a first material having a first Young's modulus;
the deformable part comprises weakening zones of the first material, arranged as a function of the first Young's modulus to bend the first material toward the piston rod according to the deflection of between 5% and 25% of the length of the piston rod, when the injection pressure is manually exerted on the manual pusher;
the deformable part of the manual pusher is able to move between a rest position and a bent position in which the deformable part is bent toward the piston rod according to the deflection of between 5% and 25% of the length of the piston rod; and
the weakening zones of the first material each extend along an axis perpendicular to the first and second longitudinal axes, when the deformable part is in the rest position.

11. The manual syringe of claim 9, wherein:
the deformable part is made of a first material having a first Young's modulus;
the deformable part comprises weakening zones of the first material, arranged as a function of the first Young's modulus to bend the first material toward the piston rod according to the deflection of between 5% and 25% of the length of the piston rod, when the injection pressure is manually exerted on the manual pusher;
the deformable part of the manual pusher is able to move between a rest position and a bent position in which the deformable part is bent toward the piston rod according to the deflection of between 5% and 25% of the length of the piston rod; and
the weakening zones of the first material have a longitudinal section, along the first longitudinal axis, defining an inverted U-shape or an inverted V-shape when the deformable part is in the rest position and when the first longitudinal axis is a vertical axis, the second end of the piston rod being situated above the first end of the piston rod with respect to the vertical axis.

12. The manual syringe of claim 10, wherein:
the manual pusher has a length along the second longitudinal axis and
the weakening zones of the first material have a total length, along the second longitudinal axis, of between 30% and 50% of the length of the manual pusher, when the deformable part is in the rest position.

13. The manual syringe of claim 9, wherein:
the manual pusher comprises an upper part on which an injection pressure suitable for injecting the product is manually exerted, and a lower part opposite the upper part; and
the lower part of the manual pusher comprises two stop members arranged on either side of the first longitudinal axis so as to block sliding of fingers along the second longitudinal axis.

14. The manual syringe of claim 9, wherein:
the manual pusher successively comprises a first lateral zone, a central zone, and a second lateral zone along the second longitudinal axis;
the central zone forms the rigid part; and
the first lateral zone and/or the second lateral zone form the deformable part.

15. The manual syringe of claim 1, wherein:
the manual pusher comprises a central zone and a peripheral zone extending around the central zone
the central zone forms the rigid part; and
the peripheral zone forms the deformable part.

16. The manual syringe of claim 1, wherein the manual pusher comprises a piston head formed at the second end of the piston rod, the piston head comprising the rigid part and the deformable part

17. The manual syringe of claim 1, wherein the manual pusher comprises:
a rigid piston head formed at the second end of the piston rod; and
a gripping member, arranged to cover the rigid piston head

18. The manual syringe of claim 17, wherein:
the gripping member comprises a rigid zone and a deformable zone;
the rigid part of the manual pusher is formed by the rigid piston head and the rigid zone of the gripping member and
the deformable part of the manual pusher is formed by the deformable zone of the gripping member.

19. The manual syringe of claim 17, wherein:
the gripping member comprises a deformable zone;
the rigid part of the manual pusher is formed by the rigid piston head and
the deformable part of the manual pusher is formed by the deformable zone of the gripping member.

20. The manual syringe of claim 1, wherein the length of the piston rod is between 40 mm and 120 mm.
